(19) United States
(12) Patent Application Publication
(10) Pub. No.: US 2006/0292105 A1
(43) Pub. Date: Dec. 28, 2006

(54) Topical preservative compositions

(76) Inventors: O. William Lever JR., Pittsford, NY (US); Erning Xia, Penfield, NY (US)

Correspondence Address: Bausch & Lomb Incorporated, One Bausch & Lomb Place, Rochester, NY, 14604-2701, US

(21) Appl. No.: 11/475,586

(22) Filed: Jun. 27, 2006

Related U.S. Application Data
(60) Provisional application No. 60/694,486, filed on Jun. 28, 2005.

(51) Int. Cl. A61K 8/43

(52) U.S. Cl. 424/70.28

(57) ABSTRACT

The use of one or more compositions of one or more preserving agents at a relatively high osmolality to disinfect contact lenses and preserve topical agents and ophthalmic lens compositions is described. Ophthalmic lens solutions containing one or more compositions of one or more preserving agents at a relatively high osmolality and methods of making and using the same are also described.

### CROSS REFERENCE

This application claims the benefit of Provisional Patent Application No. 60/694,486 filed Jun. 28, 2005 and is incorporated herein by reference.

### FIELD OF THE INVENTION

The present invention is directed toward a composition with enhanced preservation.

### BACKGROUND OF THE INVENTION

The most prevalent method of delivering an ocular pharmaceutical to the eye of a patient is to administer it topically in the form of drops, gels or ointments. To sell any topical ophthalmic pharmaceutical in the U.S for use over an extended period of time, the composition must prevent growth of microorganisms as defined in the preservative efficacy test.

In the pharmaceutical industry, benzalkonium chloride is one of the most popular preservatives for topical ophthalmic formulations. However, benzalkonium chloride can destabilize the tear film and cause irritation. Benzalkonium chloride used with a medical device such as a contact lens can potentially bind to the medical device such as a contact lens. There is a need in the pharmaceutical industry for preservatives of topically applied compositions such as ophthalmic solutions, pharmaceuticals, artificial tears and comfort drops with a gentle preservative at as low possible volumes in the topically applied compositions.

One class of solutions that are used of solution is a contact lens care solution, particularly contact lens care solutions that are not rinsed from the lens before a patient installs the lens in the eye. Multi-purpose lens care solutions both clean and disinfect the contact lens. Thus, higher levels of a disinfectant/preservative are required for multi-purpose lens care solutions. Particularly, to disinfect a lens, a disinfecting amount of a preservative/disinfectant is needed.

Potent, yet gentle, preservatives have been discovered that are sufficiently strong to disinfect a contact lens but will not cause considerable irritation when placed in the eye of the patient. U.S. Pat. No. 4,758,595 discloses a contact lens disinfectant and preservative containing a biguanide or a water-soluble salt thereof in combination with a buffer, preferably a borate buffer, e.g., boric acid, sodium borate, potassium tetraborate, potassium metaborate or mixtures of the same.

Alexidine is the trade name for 1,1′-hexamethylene-bis[5-(2-ethylhexyl)biguanide]. It is currently used as a disinfectant in a contact lens care solutions. Alexidine is known to be a gentle and potent antimicrobial agent in contact lenses.

Poly(hexamethylene biguanide) or Alexidine are listed as one of several preservatives can be used to preserve topical ophthalmic formulations. No topical ophthalmic formulations have used poly(hexamethylene biguanide) or Alexidine as a preservative have been known to date.

Nonetheless, there exists a need for a topical ophthalmic formulation that is gentle and effectively reduces the quantity of preservative that is required in solution. The present invention addresses these and other needs.

### SUMMARY OF THE INVENTION

The present invention relates to gentle preservative compositions useful for preserving topically applied agents such as ophthalmic solutions, pharmaceuticals, artificial tears and comfort drops against microbial contamination, and for preserving contact lens solutions. The subject preservative compositions are non-toxic, simple to use and do not cause ocular irritation. Additionally, preservative compositions of the present invention are suitable for use with all types of contact lenses, including rigid gas permeable contact lenses.

Compositions formulated in accordance with the present invention include a relatively lower level, or a reduced volume-preserving amount, of one or more preservative agents in solution at a relatively higher osmolality for enhanced preservation. Such solutions including one or more compositions of the present invention at a higher osmolality are useful for preserving, soaking, rinsing, wetting and conditioning all types of contact lenses, including rigid gas permeable contact lenses. The higher osmolality of solutions of the present invention allows for the use of a relatively lowered level, or a reduced volume preserving amount, of one or more preservative agents while still achieving effective preservation of pharmaceuticals, ophthalmic solutions, medical devices and the like. Due to the presence of a lower level or a reduced volume-preserving amount of one or more preserving agents, patient comfort is increased and side effects are avoided. Hence, a more gentle preservative system is achieved. By reduced volume preserving amount it is meant an amount that is less than the minimum amount required to preserve a solution at an osmolality of 220 mOsmo/Kg.

Accordingly, in one embodiment of the present invention the compositions and solutions have enhanced preserving activity useful in the manufacture of ophthalmic systems.

Accordingly, in another embodiment of the present invention there is a method for using compositions with enhanced disinfecting activity in the preservation of contact lens care solutions.

Accordingly, in one embodiment of the present invention the compositions and solutions have enhanced disinfecting activity useful in ophthalmic systems for disinfecting contact lenses.

Accordingly, in one embodiment of the present invention the compositions and solutions have enhanced preservative activity useful in preserving ophthalmic systems from microbial contamination.

Accordingly, in another embodiment of the present invention the compositions and solutions have enhanced disinfecting activity useful in ophthalmic systems for preserving contact lenses with reduced or eliminated eye irritation.

Accordingly, in still another embodiment of the present invention there is a method of making compositions with enhanced preserving activity useful in ophthalmic systems.

Accordingly, in an embodiment of the present invention there is a method of making compositions with enhanced preserving activity useful as preservative agents.

In one embodiment, there is a composition comprising water and a reduced volume preserving amount of said one or more preserving agents selected from the group consisting of poly(hexamethylene biguanide), 1,1′-hexamethylene-bis[5-(2-ethylhexyl)biguanide] and combinations thereof. The composition has a high osmolality.

In another embodiment, the composition further comprises water and a preserving amount of said one or more preserving agents selected from the group consisting of poly(hexamethylene biguanide), 1,1′-hexamethylene-bis[5-(2-ethylhexyl)biguanide] and combinations thereof. The composition further comprises a pharmaceutical agent. Optionally, the composition has a high osmolality.

These and other objectives and advantages of the present invention, some of which are specifically described and others that are not, will become apparent from the detailed description and claims that follow.

### DETAILED DESCRIPTION OF THE INVENTION

Compositions of the present invention include one or more preserving agents. Suitable preserving agents for use in the present invention include for example but are not limited to 1,1′-hexamethylene-bis[5-(p-chlorophenyl)biguanide] (Chlorhexidine) or water soluble salts thereof, 1,1′-hexamethylene-bis[5-(2-ethylhexyl)biguanide] (Alexidine) or water soluble salts

Thereof, poly(hexamethylene biguanide) (PHMB) or water-soluble salts thereof, polyquaternium-1 and quaternary ammonium compounds of low and high molecular weight. Biguanides are described in U.S. Pat. Nos. 5,990,174; 4,758,595 and 3,428,576, each incorporated herein in its entirety by reference.

The preferred preserving agents due to their ready commercial availability are poly(aminopropyl biguanide) (PAPB), also commonly referred to as poly(hexamethylene biguanide) (PHMB), and 1,1′-hexamethylene-bis[5-(2-ethylhexyl)biguanide] (Alexidine).

Compositions of one embodiment of the present invention comprise a five to thirty percent reduction in the total amount of preservative in the solution compared to the amount than is needed to establish preservative efficacy for a solution that has the same composition except that the osmolality adjusting agents are in an amount to result in an osmolality of 220 mOsmo/Kg. Since the subject compositions typically comprise five to thirty percent smaller amount of one or more preservative agents, it is surprising that the subject compositions exhibit enhanced preservative effect. For purposes of the present invention, “enhanced preservative activity” is defined as effective preservation with lower levels or reduced amounts of one or more preserving agents than in a comparable solution with an osmolality of 220 mOsmo/kg. For purposes of the present invention, “enhanced preservative activity” is defined as effective preservation with lower levels or reduced amounts of one or more preserving agents than in a comparable solution except that the amount of osmolality adjusting agent is adjusted to result in an osmolality of 220 mOsmo/Kg. Standard total amounts of preserving agents in commercially available lens care solutions are in the range of 0.5 parts per million (ppm) to 15 ppm. Accordingly, compositions of the present invention comprise about 0.35 ppm to about 14.25 ppm, and more preferably about 0.35 ppm to about 10.5 ppm of one or more preserving agents. In one embodiment, the present invention comprises from about 1 ppm to about 5 ppm. Compositions of the present invention may also be used in conjunction with other known preserving agents if desired. Compositions of the present invention are preferably in solution in sufficient concentration to prevent microorganisms and thus preserve the solution from microbial contamination throughout the intended shelf life of the solution.

One or more compositions of the present invention are used in solutions having a relatively higher osmolality. By higher osmolality it is meant a solution that is hypertonic. In one embodiment, the osmolality of the compositions or solutions of the present invention is a minimum of about 280 mOsmo/Kg, about 285 mOsmo/Kg, about 290 mOsmo/Kg, about 300 mOsmo/Kg, about 310 mOsmo/Kg, about 320 mOsmo/Kg, about 330 mOsmo/Kg and a maximum of about 400 mOsmo/Kg, about 380 mOsmo/Kg, about 360 mOsmo/Kg, about 340 mOsmo/Kg. Such solutions of the present invention typically comprise an increase in osmolality over commercially sold product that is a minimum of about five percent, about 10 percent, about 15 percent and is a maximum of about 25 percent, about 20 percent or about 15 percent. Compositions of the present invention likewise include one or more tonicity-adjusting agents. Examples of suitable tonicity agents include but are not limited to sodium and potassium chloride, dextrose, mannose, glycerin, propylene glycol, calcium and magnesium chloride.

Compositions of the present invention in solution are physiologically compatible or “ophthalmically safe” for use with contact lenses. Ophthalmically safe as used herein means that a contact lens treated with or in the subject solution is generally suitable and safe for direct placement on the eye without rinsing. The subject solutions are safe and comfortable for daily contact with the eye via a contact lens that has been wetted with the solution. An ophthalmically-safe solution has a tonicity and pH that is compatible with the eye and comprises materials, and amounts thereof, that are non-cytotoxic according to ISO International Standards Organization) standards and U.S. FDA (Food and Drug Administration) regulations. The solution should be sterile in that the absence of microbial contaminants in the product prior to release should be statistically demonstrated to the degree necessary for such products.

Compositions of the present invention can be used with all contact lenses such as conventional hard and soft lenses, as well as rigid and soft gas permeable lenses. Such suitable lenses include both hydrogel and non-hydrogel lenses, as well as silicone and fluorine-containing lenses. The term “soft contact lens” as used herein generally refers to those contact lenses that readily flex under small amounts of force. Typically, soft contact lenses are formulated from polymers having a certain proportion of repeat units derived from monomers such as 2-hydroxyethyl methacrylate and/or other hydrophilic monomers, typically cross-linked with a cross-linking agent. However, newer soft lenses, especially for extended wear, are being made from high-Dk silicone-containing materials.

Compositions of the present invention when in solution likewise include one or more buffers or a buffering system, in addition to or in place of the aminoalcohol buffer, to adjust the final pH of the solution. Suitable buffers include for example but are not limited to phosphate buffers, borate buffers, tris(hydroxymethyl)aminomethane (Tris) buffers, bis(2-hydroxyethyl)imino-tris(hydroxymethyl)methane (bis-Tris) buffers, citrate buffers, sodium bicarbonate, and combinations thereof. A suitable buffering system for example may include at least one phosphate buffer and at least one borate buffer, which buffering system has a buffering capacity of 0.01 to 0.5 mM, preferably 0.03 to 0.45, of 0.01 N of HCl and 0.01 to 0.3, preferably 0.025 to 0.25, of 0.01 N of NaOH to change the pH one unit. Buffering capacity is measured by a solution of the buffers only.

Compositions of the present invention may optionally include one or more aminoalcohol buffers, such as ethanolamine buffers, present in a total amount of from approximately 0.02 to approximately 3.0 percent by weight based on the total weight of the composition. Suitable aminoalcohol buffers include for example but are not limited to monoethanolamine (MEA), diethanolamine (DEA), triethanolamine (TEA), 2-amino-2-methyl-1,3-propanediol (AMPD), 2-dimethylamino-2-methyl-1-propanediol (DMAMP), 2-amino-2-ethylpropanol (AEP), 2-amino-1-butanol (AB) and 2-amino-2-methyl-1-propanol (AMP), but preferably MEA, DEA or TEA.

Compositions of the present invention may optionally include one or more cationic polysaccharides. One or more cationic polysaccharides are present in the subject compositions in a total amount of from approximately 0.001 to approximately 0.5 percent by weight based on the total weight of the composition, but more preferably from about 0.005 to about 0.05 percent by weight. Suitable cationic polysaccharides for use in compositions of the present invention include for example but are not limited to variations of polyquaternium-10 such as for example but not limited to Polymer JR 125™ (Dow Chemical Company, Midland, Mich.) having a 2 percent solution viscosity of 75-125 cPs and 1.5 to 2.2 percent nitrogen, Polymer JR 400™ (Dow Chemical Company) having a 2 percent solution viscosity of 300 to 500 cPs and 1.5 to 2.2 percent nitrogen, Polymer JR 30M™ (Dow Chemical Company) having a 1 percent solution viscosity of 1,000 to 2,500 cPs and 1.5 to 2.2 percent nitrogen, Polymer LR 400™ (Dow Chemical Company) having a 2 percent solution viscosity of 300 to 500 cPs and 0.8 to 1.1 percent nitrogen, Polymer LR 30M™ (Dow Chemical Company) having a 1 percent solution viscosity of 1,250 to 2,250 cPs and 0.8 to 1.1 percent nitrogen, and Polymer LK™ (Dow Chemical Company) having a 2 percent solution viscosity of 300 to 500 cPs and 0.8 to 1.1 percent nitrogen. The preferred cationic polysaccharide for use in the present invention is Polymer JR 125™ or Polymer JR 400™.

Compositions of the present invention may likewise optionally include one or more surfactants that have known advantages in terms of cleaning efficacy and comfort. Surfactants are present in the subject compositions in a total amount of from approximately 0.001 to approximately 25.0 percent by weight based on the total weight of the composition, but more preferably from about 0.001 to about 5.0 percent by weight. Suitable surfactants include for example but are not limited to sugar-containing polyethers, aliphatic or aromatic hydrocarbon-containing polyethers, polyethers based upon poly(ethylene oxide)-poly(propylene oxide)-poly(ethylene oxide), i.e., (PEO-PPO-PEO), or poly(propylene oxide)-poly(ethylene oxide)-poly(propylene oxide), i.e., (PPO-PEO-PPO), or a combination thereof. PEO-PPO-PEO and PPO-PEO-PPO are commercially available under the trade names Pluronics™, R-Pluronics™, Tetronics™ and R-Tetronics™ (BASF Wyandotte Corp., Wyandotte, Mich.) and are further described in U.S. Pat. No. 4,820,352 incorporated herein in its entirety by reference. Suitable surfactants for use in the present composition should be soluble in the lens care solution, not become turbid, and should be non-irritating to eye tissues.

Optionally, it may also be desirable to include one or more water-soluble viscosity agents in the subject composition. Because of the demulcent effect of viscosity agents, the same have a tendency to enhance the lens wearer's comfort by means of a film on the lens surface cushioning impact against the eye. Suitable viscosity agents include for example but are not limited to water-soluble cellulose polymers like hydroxyethyl cellulose, hydroxypropyl cellulose or carboxymethyl cellulose, povidone, poly(vinyl alcohol), poly(ethylene glycol), poly(ethylene oxide) and the like. Viscosity agents may be employed in amounts ranging from about 0.01 to about 4.0 weight percent or less.

Compositions of the present invention may optionally include one or more sequestering agents to bind metal ions, which in the case of ophthalmic solutions, might otherwise react with protein deposits and collect on contact lenses. Suitable sequestering agents include for example but are not limited to ethylenediaminetetraacetic acid (EDTA) and its salts. Another useful class of sequestering agents are phosphonates or hydroxyalkylphosphonates (HAP), such as those disclosed in U.S. Pat. No. 5,858,937 (Richards et al.), available under the trade name Dequest® (Monsanto Co., St. Louis, Mo.), and most preferably tetra sodium etidronate available under the trade name Dequest® 2016 (Monsanto Co.). Sequestering agents are preferably used in amounts ranging from about 0.01 to about 0.2 weight percent.

Optionally, the composition contains a pharmaceutical agent. By pharmaceutical agent it is meant a pharmaceutically active agent that relieves a medical condition. The pharmaceutical agent of one embodiment is an agent that falls within one of the therapeutic classes below including any one or more of the specific pharmaceutical agents listed below:

Anti-Metabolites:

Examples of anti-metabolites include, but are not limited to, folic acid analogs (e.g., denopterin, edatrexate, methotrexate, piritrexim, pteropterin, Tomudex[R], trimetrexate), purine analogs (e.g., cladribine, fludarabine, 6-mercaptopurine, thiamiprine, thiaguanine) and pyrimidine analogs (e.g., ancitabine, azacitidine, 6-azauridine, carmofur, cytarabine, doxifluridine, emitefur, enocitabine, floxuridine, fluorouracil, gemcitabine, tegafur).

Anti-biotics:

Specific antibiotics that, optionally, are useful in combination with one or more fused pyrrolocarbazole of the present invention include but are not limited to aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin sv, rifapentine, rifaximin), β-lactams (e.g., carbacephems (e.g., loracarbef), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefinenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefinetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, aziocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, meziocillin, nafcillin sodium, oxacillin, penamecillin, penethamate hydriodide, penicillin g benethamine, penicillin g benzathine, penicillin g benzhydrylamine, penicillin g calcium, penicillin g hydrabamine, penicillin g potassium, penicillin g procaine, penicillin n, penicillin o, penicillin v, penicillin v benzathine, penicillin v hydrabamine, penimepicycline, phenethicillin potassium, piperacillin, pivampicillin, propicillin, quinacillin, sulbenicillin, sultamicillin, talampicillin, temocillin, ticarcillin), other (e.g., ritipenem), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline) and others (e.g., cycloserine, mupirocin, tuberin).

Synthetic Antibacterials:

Specific anti-bacterial agents that, optionally, are useful in combination with one or more embodiment of the present invention include but are not limited to 2,4-Diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-b, chloramine-t, dichloramine t, n₂-formylsulfisomidine, n (4)-β-d-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, 4-sulfanilamidosalicylic acid, n (4)-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole)sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone) and others (e.g., clofoctol, hexedine, methenamine, methenamine anhydromethylene-citrate, methenamine hippurate, methenamine mandelate, methenamine sulfosalicylate, nitroxoline, taurolidine, xibomol).

Antifungal Antibiotics:

Specific antifungal antibiotics that, optionally, are useful in combination with one or more embodiment of the present invention include but are not limited to polyenes (e.g., amphotericin b, candicidin, dermostatin, filipin, fungichromin, hachimycin, hamycin, lucensomycin, mepartricin, natamycin, nystatin, pecilocin, perimycin), others (e.g., azaserine, griseofulvin, oligomycins, neomycin undecylenate, pyrroInitrin, siccanin, tubercidin, viridin).

Synthetic Antifungals:

Specific synthetic antifungal agents that, optionally, are useful in combination with one or more embodiment of the present invention include but are not limited to allylamines (e.g., butenafine, naftifine, terbinafine), imidazoles (e.g., bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole) others (e.g., acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, zinc propionate).

Steroids:

Specific steroidal anti-inflammatory agents, optionally, are useful in combination with one or more pharmaceutical agent of the present invention include but are not limited to glucocorticoids. Non-limiting illustrative specific examples of steroidal anti-inflammatory agents include the following medicaments: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, methylprednisolone aceponate, mometasone furoate, paramethasone, prednicarbate, prednisolone, prednisolone 25-diethylaminoacetate, prednisolone sodium phosphate, prednisone, prednival, prednylidene, rimexolone, rofleponide, tixocortol, triamcinolone, triamcinolone acetonide, triamcinolone benetonide, and triamcinolone hexacetonide, salts of any of these compounds, esters of any of these compounds and combinations thereof.

Anti-Proliferative Agents:

Specific examples of anti-proliferative agents that, optionally, are useful, in combination with one or more fused pyrrolocarbazoles of the present invention include angiopeptin, angiotensin converting enzyme inhibitors such as captopril, cilazapril or lisinopril; calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug), monoclonal antibodies (such as those specific for Platelet-Derived Growth Factor (PDGF) receptors), nitroprusside, phosphodiesterase inhibitors, prostaglandin inhibitors, suramin, seratonin blockers, steroids, thioprotease inhibitors, triazolopyrimidine (a PDGF antagonist), and nitric oxide.

Matrix Metalloproteinase Inhibitors:

Specific examples of matrix metalloproteinase inhibitors that are optionally useful in combination with one or more fused pyrrolocarbazoles of the present invention include but are not limited to prinomastat, ilomastat, marimastat and batimastat.

Thrombolytic Agents:

Specific examples of thrombolytic agents that are optionally useful in combination with one or more fused pyrrolocarbazoles of the present invention include urokinase, streptokinase, tissue plasminogen activator (TPA) or a similar therapeutic species.

Anti-Neoplastic Agents:

Specific examples of anti-neoplastic agents that are useful in combination with one or more fused pyrrolocarbazoles of the present invention include antibiotics and analogs (e.g., aclacinomycin, actinomycin, anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycines, peplomycin, pirarubicin, plicamycin, porfiromycin, puromycin, streptonigrin, streptozocin, tubercidin, zinostatin, zorubicin) and antimetabolites (e.g. folic acid analogs, denopterin, edatrexate, methotrexate, piritrexim, pteropterin, Tomudex®, trimetrexate, purine analogs, cladribine, fludarabine, 6-mercaptopurine, thiamiprine, thioguanine, pyrimidine analogs, ancitabine, azacitidine, 6-azauridine, carmofur, cytarabine, doxifluridine, emitefur, enocitabine, floxuridine, fluorouracil, gemcitabine, tagafur).

Non-Steriodal Anti-Inflammatory Drugs (NSAIDS):

Non-steroidal anti-inflammatory drugs (NSAIDS) that are useful optionally in combination with one or more fused pyrrolocarbazole of the present include but are not limited to ibuprofen, nimesulide, diclofenac and its alkali metal salts; fenoprofen and its metal salts; fluriprofen; ketoprofen; naproxen and its alkali metal salts; nimesulide; and piroxicam and its salts.

Retinoids:

Examples of retinoids that optionally are useful in combination with one or more fused pyrrolocarbazole of the present invention include but are not limited to retinoic acid, N-(4-hydroxyphenl) retinamide-O-glucuronide, N-(4-hydroxyphenyl) retinamide, O-glucuronide conjugates of retinoids, N-(4-hydroxyphenyl) retinamide and its glucuronide derivative, retinyl-B-glucuronide, the glucuronide conjugates of retinoic acid and retinol, tretinoin, etretinate, arotinoid, isotretinoin, retinyl acetate, acitretin, adapalene, and tazarotene.

The compositions of the present invention are described in still greater detail in the examples that follow.

### EXAMPLE 1

### Effect of Osmolality on Biocidal Efficacy

Sample solutions for testing the effect of osmolality on biocidal efficacy, were prepared in accordance with the formulations set forth below in Table 1.

TABLE 1
Test Sample Solutions

| Ingredients | Sample | | | | |
|---|---|---|---|---|---|
| W/W Percent | 1 | 2 | 3 | 4 | 5 |
| Sodium Borate | 0.09 | 0.09 | 0.09 | 0.09 | 0.09 |
| Boric Acid | 0.85 | 0.85 | 0.85 | 0.85 | 0.85 |
| Sodium Chloride | 0.22 | 0.22 | 0.22 | 0.22 | 0.45 |
| Alexidine 2HCl (ppm) | 0.1 | 0.2 | 0.3 | 0.4 | 0.1 |
| PHMB HCl (ppm) | 0 | 0 | 0 | 0 | 0 |
| pH | 7.0 | 7.0 | 7.0 | 7.0 | 7.0 |
| Osmolality (mOsmo/Kg) | 220 | 220 | 220 | 220 | 300 |
| Ingredients | Sample | | | | |
| w/w Percent | 6 | 7 | 8 | 9 | 10 |
| Sodium Borate | 0.09 | 0.09 | 0.09 | 0.09 | 0.09 |
| Boric Acid | 0.85 | 0.85 | 0.85 | 0.85 | 0.85 |
| Sodium Chloride | 0.45 | 0.45 | 0.45 | 0.22 | 0.45 |
| Alexidine 2HCl (ppm) | 0.2 | 0.3 | 0.4 | 0 | 0 |
| PHMB HCl (ppm) | 0 | 0 | 0 | 0.1 | 0.2 |
| pH | 7.0 | 7.0 | 7.0 | 7.0 | 7.0 |
| Osmolality (mOsmo/Kg) | 300 | 300 | 300 | 220 | 220 |
| Ingredients | Sample | | | | |
| w/w Percent | 11 | 12 | 13 | 14 | 15 |
| Sodium Borate | 0.09 | 0.09 | 0.09 | 0.09 | 0.09 |
| Boric Acid | 0.85 | 0.85 | 0.85 | 0.85 | 0.85 |
| Sodium Chloride | 0.22 | 0.22 | 0.45 | 0.45 | 0.45 |
| Alexidine 2HCl (ppm) | 0 | 0 | 0 | 0 | 0 |
| PHMB HCl (ppm) | 0.3 | 0.4 | 0.1 | 0.2 | 0.3 |
| pH | 7.0 | 7.0 | 7.0 | 7.0 | 7.0 |
| Osmolality (mOsmo/Kg) | 220 | 220 | 300 | 300 | 300 |
| Ingredients | Sample | | | | |
| w/w Percent | 16 | 17 | 18 | 19 | 20 |
| Sodium Borate | 0.09 | 0.09 | 0.09 | 0.09 | 0.09 |
| Boric Acid | 0.85 | 0.85 | 0.85 | 0.85 | 0.85 |
| Sodium Chloride | 0.45 | 0.22 | 0.22 | 0.45 | 0.45 |
| Alexidine 2HCl (ppm) | 0 | 0.1 | 0.2 | 0.1 | 0.2 |
| PHMB HCl (ppm) | 0.4 | 0.1 | 0.2 | 0.1 | 0.2 |
| pH | 7.0 | 7.0 | 7.0 | 7.0 | 7.0 |

Test solutions prepared in accordance with the formulations set forth above in Table 1, were each tested to determine the effect of osmolality on biocidal efficacy. Test solutions prepared in accordance with Table 1 above, were tested for ISO/FDA microbial biocidal efficacy using five FDA/ISO challenge microorganisms, i.e., three bacteria and two fungi. Primary acceptance criteria established for bacteria require that the number of viable bacteria, recovered per ml, shall be reduced by a value not less than 3.0 logs at 14 days. After the rechallenge at day 14, the concentration of bacteria shall be reduced by at least 3.0 logs by day 28. Primary acceptance criteria established for yeasts and molds require that the number of viable yeasts and molds, recovered per ml, remain at or below initial concentrations within an experimental error of ±0.5 logs within 14 days. After day 28, the concentration of mold and yeast shall remain at or below the concentrations after rechallenge within an experimental error ±0.5 logs. of shall be reduced by a value of not less than 1.0 logs within the minimum recommended disinfection time with no increase at not less than four times the minimum recommended disinfection time within an experimental error of +/−0.5 logs. Results of the ISO/FDA microbial preservative efficacy testing of the subject test solutions are set forth below in Table 2. The results set forth in Table 2 illustrate that lower volumes of preserving agent(s) are necessary at higher osmolalities to achieve effective solution preservation.

TABLE 2
Biocidal Efficacy With Varying Osmolality

| | | Log Reduction of Sample | | | | |
|---|---|---|---|---|---|---|
| ISO Agent | Days | 1 | 2 | 3 | 4 | 5 |
| Staphylococcus aureus | | | | | | |
| (ATCC 6538) | 7 | 3.1 | >4.7 | >4.7 | >4.7 | 1.7 |
| | 14 | >4.7 | >4.7 | >4.7 | >4.7 | >4.7 |
| | 21 | 1.0 | 2.7 | >3.9 | >3.9 | 1.1 |
| | 28 | >3.9 | >3.9 | >3.9 | >3.9 | >3.9 |
| Pseudomonas aeruginosa | | | | | | |
| (ATCC 9027) | 7 | 2.1 | 4.8 | >4.8 | >4.8 | 1.8 |
| | 14 | 3.2 | >4.8 | >4.8 | >4.8 | 3.2 |
| | 21 | 1.2 | >3.7 | >3.7 | >3.7 | 1.6 |
| | 28 | 2.1 | >3.7 | >3.7 | >3.7 | 3.3 |
| Escherichia coli | | | | | | |
| (ATCC 8739) | 7 | 2.3 | 3.2 | 3.5 | 4.2 | 1.7 |
| | 14 | 2.8 | >4.7 | >4.7 | >4.7 | 2.6 |
| | 21 | ND | 2.6 | >3.7 | >3.7 | ND |
| | 28 | 1.1 | >3.7 | >3.7 | >3.7 | 1.0 |
| Candida albicans | | | | | | |
| (ATCC 10231) | 7 | 0.3 | 0.6 | 1.3 | 4.2 | 0.4 |
| | 14 | 1.4 | 2.3 | 3.8 | >4.6 | 1.8 |
| | 21 | 0.5 | 0.4 | 1.1 | 3.4 | 1.0 |
| | 28 | 2.6 | 2.7 | 3.5 | >3.8 | 2.7 |
| Aspergillus niger | | | | | | |
| (ATCC 16404) | 7 | 1.2 | 1.4 | 1.4 | 1.6 | 1.5 |
| | 14 | 1.2 | 1.5 | 1.4 | 1.4 | 1.2 |
| | 21 | 0.2 | 0.0 | 0.1 | 0.0 | 0.5 |
| | 28 | 0.4 | 0.2 | 0.7 | 0.5 | 0.5 |
| | | Log Reduction of Sample | | | | |
| ISO Agent | Days | 6 | 7 | 8 | 9 | 10 |
| Staphylococcus aureus | | | | | | |
| (ATCC 6538) | 7 | >4.7 | >4.7 | >4.7 | 2.1 | >4.7 |
| | 14 | >4.7 | >4.7 | >4.7 | >4.7 | >4.7 |
| | 21 | 1.2 | 3.7 | >3.9 | 1.0 | 2.7 |
| | 28 | 3.6 | >3.9 | >3.9 | >3.9 | >3.9 |
| Pseudomonas aeruginosa | | | | | | |
| (ATCC 9027) | 7 | 4.8 | >4.8 | 4.8 | 1.3 | 4.8 |
| | 14 | >4.8 | >4.8 | >4.8 | 2.4 | >4.8 |
| | 21 | >3.7 | >3.7 | >3.7 | ND | >3.7 |
| | 28 | >3.7 | >3.7 | >3.7 | 1.3 | >3.7 |
| Escherichia coli | | | | | | |
| (ATCC 8739) | 7 | 3.5 | 3.2 | 3.8 | 2.2 | 3.2 |
| | 14 | >4.7 | >4.7 | >4.7 | 2.9 | >4.7 |
| | 21 | 2.9 | 3.2 | 3.3 | 1.4 | 2.6 |
| | 28 | >3.7 | >3.7 | >3.7 | 1.4 | >3.7 |
| Candida albicans | | | | | | |
| (ATCC 10231) | 7 | 0.8 | 1.2 | 4.3 | 0.3 | 0.6 |
| | 14 | 2.5 | 2.8 | >4.6 | 1.4 | 2.3 |
| | 21 | 1.1 | 1.3 | 3.2 | 0.7 | 0.4 |
| | 28 | >3.8 | 3.6 | >3.8 | 2.8 | 2.7 |
| Aspergillus niger | | | | | | |
| (ATCC 16404) | 7 | 1.8 | 1.6 | 1.4 | 1.5 | 1.4 |
| | 14 | 1.3 | 1.3 | 1.3 | 1.4 | 1.5 |
| | 21 | 0.1 | 0.1 | 0.0 | 0.1 | 0.0 |
| | 28 | 0.3 | 0.6 | 0.5 | 0.5 | 0.2 |
| | | Log Reduction of Sample | | | | |
| ISO Agent | Days | 11 | 12 | 13 | 14 | 15 |
| Staphylococcus aureus | | | | | | |
| (ATCC 6538) | 7 | >4.7 | >4.7 | 2.3 | 4.0 | >4.7 |
| | 14 | >4.7 | >4.7 | >4.7 | >4.7 | >4.7 |
| | 21 | 3.7 | >3.9 | 0.8 | 1.6 | 3.2 |
| | 28 | >3.9 | >3.9 | 3.9 | >3.9 | >3.9 |
| Pseudomonas aeruginosa | | | | | | |
| (ATCC 9027) | 7 | >4.8 | >4.8 | 1.3 | 4.3 | >4.8 |
| | 14 | >4.8 | >4.8 | 2.4 | >4.8 | >4.8 |
| | 21 | >3.7 | 3.7 | ND | 2.6 | >3.7 |
| | 28 | >3.7 | >3.7 | 1.5 | >3.7 | >3.7 |
| Escherichia coli | | | | | | |
| (ATCC 8739) | 7 | >4.7 | >4.7 | 2.2 | 3.8 | 4.7 |
| | 14 | >4.7 | >4.7 | 3.2 | 3.9 | >4.7 |
| | 21 | 2.9 | >3.7 | ND | 2.3 | 3.2 |
| | 28 | >3.7 | >3.7 | 1.0 | 3.7 | >3.7 |
| Candida albicans | | | | | | |
| (ATCC 10231) | 7 | 2.5 | 4.4 | 0.4 | 2.7 | 3.4 |
| | 14 | >4.6 | >4.6 | 2.3 | >4.6 | >4.6 |
| | 21 | 1.3 | 2.5 | 0.8 | 0.9 | 1.4 |
| | 28 | >3.8 | >3.8 | 3.1 | >3.8 | >3.8 |
| Aspergillus niger | | | | | | |
| (ATCC 16404) | 7 | 1.5 | 1.5 | 1.7 | 1.5 | 1.7 |
| | 14 | 1.2 | 1.3 | 1.3 | 1.4 | 1.3 |
| | 21 | 0.3 | 0.3 | 0.1 | 0.1 | 0.3 |
| | 28 | 0.6 | 0.7 | 0.7 | 0.7 | 0.6 |
| | | Log Reduction of Sample | | | | |
| ISO Agent | Days | 16 | 17 | 18 | 19 | 20 |
| Staphylococcus aureus | | | | | | |
| (ATCC 6538) | 7 | >4.7 | 4.5 | >4.7 | 3.4 | >4.7 |
| | 14 | >3.9 | >4.7 | >4.7 | >4.7 | >4.7 |
| | 21 | >3.9 | 1.4 | >3.9 | 0.9 | >3.9 |
| | 28 | >4.8 | >3.9 | >3.9 | 3.9 | >3.9 |
| Pseudomonas aeruginosa | | | | | | |
| (ATCC 9027) | 7 | >4.8 | 3.1 | >4.8 | 4.8 | >4.8 |
| | 14 | >4.8 | 4.1 | >4.8 | 4.1 | >4.8 |
| | 21 | >3.7 | 1.4 | >3.7 | 2.8 | >3.7 |
| | 28 | >3.7 | 2.5 | >3.7 | >3.7 | >3.7 |
| Escherichia coli | | | | | | |
| (ATCC 8739) | 7 | >4.7 | 3.5 | 4.5 | 3.2 | 4.5 |
| | 14 | >4.7 | 4.2 | >4.7 | >4.7 | >4.7 |
| | 21 | >3.7 | 1.1 | 3.2 | 3.4 | 3.2 |
| | 28 | >3.7 | 1.4 | >3.7 | >3.7 | >3.7 |
| Candida albicans | | | | | | |
| (ATCC 10231) | 7 | 1.3 | 0.7 | 4.6 | 4.6 | 4.6 |
| | 14 | 3.8 | 2.3 | >4.6 | >4.6 | >4.6 |
| | 21 | 1.1 | 0.5 | 1.6 | 1.6 | 1.8 |
| | 28 | 3.5 | 2.7 | >3.8 | >3.8 | >3.8 |
| Aspergillus nigeri | | | | | | |
| (ATCC 16404) | 7 | 1.4 | 1.4 | 1.3 | 1.3 | 1.5 |
| | 14 | 1.4 | 1.4 | 1.4 | 1.4 | 1.2 |
| | 21 | 0.1 | 0.2 | 0.1 | 0.1 | 0.1 |
| | 28 | 0.7 | 0.3 | 0.5 | 0.5 | 0.6 |

One or more preserving agent-containing compositions of the present invention are useful as contact lens care solutions for disinfecting contact lenses. An effective preserving amount of preserving agent is an amount that will at least partially reduce the microorganism population in the formulations employed. Preferably, a preserving amount is that which will reduce the microbial burden of representative bacteria by two log orders in four hours and more preferably by one log order in one hour. Most preferably, a preserving amount is an amount that will eliminate the microbial burden on a contact lens when used according to its regimen for the recommended soaking time as established by ISO (International Standards for Ophthalmic Optics)/FDA Stand-Alone Procedures for Disinfection Test (ISO/DIS 14729; 2001). Typically, such agents are present in concentrations ranging from about 0.00001 to about 0.5 percent weight/volume (w/v), and more preferably, from about 0.00003 to about 0.5 percent w/v. Unexpectedly, when used at a high osmolality, i.e., above about 200 mOsmo/Kg, a smaller amount of preserving agent, i.e., a 5 to 30 percent reduction and more preferably a 15 to 30 percent reduction, is required to achieve effective solution preservation.

As stated above, contact lenses or similar medical devices are preserved by contacting the lens or device with a solution of one or more compositions of the present invention. Although this may be accomplished by simply soaking a lens in the subject solution, if desired, a few drops of the solution may be initially placed on each side of the lens prior to rubbing the lens for a period of time, for example, approximately 20 seconds. The lens can then be subsequently immersed within several milliliters of the subject solution. Preferably, the lens is permitted to soak in the solution for at least four hours. The lenses are then removed from the solution, rinsed with the same or a different solution, for example a preserved isotonic saline solution and then replaced on the eye.

Solutions containing one or more compositions of the present invention may be formulated into specific contact lens care products for use as customary in the field of ophthalmology. Such products include but are not limited to wetting solutions, soaking solutions, cleaning and conditioning solutions, as well as multipurpose type lens care solutions and in-eye cleaning and conditioning solutions.

While the invention has been described in conjunction with specific examples thereof, this is illustrative only. Accordingly, many alternatives, modifications, and variations will be apparent to those skilled in the art in the light of the foregoing description and it is, therefore, intended to embrace all such alternatives, modifications, and variations as to fall within the spirit and scope of the appended claims.

We claim:

1. A composition comprising:
a reduced volume preserving amount of the combination of poly(hexamethylene biguanide) and Alexidine at a high osmolality.

2. The composition of claim 1 wherein said high osmolality is above about 285 mOsm/kg.

3. The composition of claim 1 wherein said composition is hypertonic.

4. The composition of claim 1 wherein said solution has an osmolality that is a minimum of about 290 and a maximum of about 380 mOsmo/Kg.

5. The composition of claim 1 wherein said reduced volume preserving amount of said one or more preserving agents is a standard volume reduced by 5 to 30 percent.

6. A method of producing the composition of claim 1 comprising:
combining a reduced volume preserving amount of one or more preserving agents at a high osmolality.

7. The method of claim 6 wherein the composition has an osmolality above about 285 mOsmo/Kg.

8. The method of claim 6 wherein said composition is hypertonic.

9. The method of claim 8 wherein said composition has an osmolality that is a minimum of about 290 and a maximum of about 380 mOsmo/Kg.

10. The method of claim 8 wherein said reduced volume preserving amount of said one or more preserving agents is a standard volume reduced by 5 to 30 percent.

11. A solution comprising one or more compositions of claim 1.

12. The solution of claim 11 wherein said osmolality is a minimum of about 230 mOsmo/Kg.

13. The solution of claim 12 wherein said solution is hypertonic.

14. The solution of claim 12 wherein said solution has an osmolality that is a minimum of about 290 and a maximum of about 380 mOsmo/Kg.

15. The solution of claim 12 wherein said solution includes one or more buffers or buffering systems.

16. The solution of claim 12 wherein said solution includes one or more buffers or buffering systems selected from the group consisting of phosphate buffers, borate buffers, tris(hydroxymethyl)aminomethane (Tris) buffers, bis(2-hydroxyethyl)imino-tris(hydroxymethyl)methane (bis-Tris) buffers, sodium bicarbonate, citrate buffers, aminoalcohol buffers and combinations thereof.

17. The solution of claim 12 wherein said solution includes one or more tonicity agents.

18. The solution of claim 12 wherein said solution includes one or more tonicity agents selected from the group consisting of sodium chloride, potassium chloride, dextrose, mannose, glycerin, propylene glycol, calcium chloride and magnesium chloride.

19. The solution of claim 12 wherein said solution includes one or more surfactants.

20. The solution of claim 12 wherein said solution includes one or more surfactants selected from the group consisting of polyethers based upon poly(ethylene oxide)-poly(propylene oxide)-poly(ethylene oxide), poly(propylene oxide)-poly(ethylene oxide)-poly(propylene oxide), sugar-containing polyethers, aliphatic or aromatic hydrocarbon-containing polyethers and combinations thereof.

21. The solution of claim 12 wherein said solution includes one or more viscosity agents.

22. The solution of claim 12 wherein said solution includes one or more viscosity agents selected from the group consisting of water-soluble cellulose polymers, povidone, poly(vinyl alcohol), poly(ethylene glycol) and poly(ethylene oxide).

23. The solution of claim 12 wherein said solution includes one or more aminoalcohol buffers.

24. The solution of claim 12 wherein said solution includes one or more aminoalcohol buffers selected from the group consisting of monoethanolamine, diethanolamine, triethanolamine, 2-amino-2-methyl-1,3-propanediol, 2-dimethylamino-2-methyl-1-propanediol, 2-amino-2-ethylpropanol, 2-amino-1-butanol and 2-amino-2-methyl-1-propanol.

25. The solution of claim 12 wherein said solution includes one or more cationic polysaccharides.

26. The solution of claim 12 wherein said solution includes one or more cationic polysaccharides selected from the group consisting of variations of polyquaternium-10.

27. The solution of claim 12 wherein said solution includes one or more sequestering agents.

28. The solution of claim 13 wherein said solution includes one or more sequestering agents selected from the group consisting of phosphonates, ethylenediaminetetraacetic acid and salts of ethylenediaminetetraacetic acid.

29. A method of using the solution of claim 12 comprising:
contacting a surface of a contact lens with said solution for a period of time suitable to eliminate a microbial burden on said contact lens.

30. A method of producing the solution of claim 12 comprising:
adding a reduced volume preserving amount of said one or more preserving agents to a solution at a high osmolality.

31. An improved preserving composition including a combination of alexidine and poly(hexamethylene biguanide) in solution wherein the improvement comprises:
a reduced volume preserving amount of said one or more preserving agents at a high osmolality to achieve increased biocidal efficacy.

32. The improved composition of claim 31 wherein said high osmolality is above about 285 mOsmo/Kg.

33. The improved composition of claim 31 wherein said high osmolality is hypertonic.

34. The improved composition of claim 31 wherein said high osmolality is from about 290 to about 380 mOsmo/Kg.

35. The improved composition of claim 35 wherein said one or more preserving agents include 1,1′-hexamethylene-bis[5-(p-chlorophenyl)biguanide] or salts thereof.

36. The improved composition of claim 35 wherein said reduced volume preserving amount of said one or more preserving agents is a standard volume reduced by 5 to 30 percent.

37. A composition comprising water and a reduced volume preserving amount of said one or more preserving agents selected from the group consisting of poly(hexamethylene biguanide), 1,1′-hexamethylene-bis[5-(2-ethylhexyl)biguanide] and combinations thereof, wherein the composition has a high osmolality.

38. A composition comprising:
water;
a pharmaceutical agent; and
a preserving amount of said one or more preserving agents selected from the group consisting of poly(hexamethylene biguanide), 1,1′-hexamethylene-bis[5-(2-ethylhexyl)biguanide] and combinations thereof.

39. A composition comprising:
water;
a pharmaceutical agent; and
a reduced volume preserving amount of said one or more preserving agents selected from the group consisting of poly(hexamethylene biguanide), 1,1′-hexamethylene-bis[5-(2-ethylhexyl)biguanide] and combinations thereof, wherein the composition has a high osmolality.
